(19) United States
(12) Patent Application Publication
(10) Pub. No.: US 2012/0300171 A1
(43) Pub. Date: Nov. 29, 2012

(54) Programmable Ophthalmic Lenses

(75) Inventors: Amitava Gupta, Roanoke, VA (US); Ronald Blum, Roanoke, VA (US); William Kokonaski, Gig Harbor, WA (US)

(73) Assignee: PixelOptics, Inc., Roanoke, VA (US)

(21) Appl. No.: 13/482,280

(22) Filed: May 29, 2012

Related U.S. Application Data
(60) Provisional application No. 61/490,938, filed on May 27, 2011.

(51) Int. Cl. G02C 7/08; A61F 2/16

(52) U.S. Cl. 351/159.4; 623/6.22

(57) ABSTRACT

Ophthalmic lenses are described including a deformable layer and a deformable membrane disposed opposite the deformable layer. The lens is configured with at least two regions of adjustable optical power, such as by using a patterned electrode which is used to drive the membrane move axially along an optical path of the lens. A surface of the deformable layer is configured to expand and/or contract based on movement of the membrane along the optical path of the lens, such as by bonding one side of the deformable layer to the membrane and bonding the other side to a fixed optical element layer.

### CROSS-REFERENCES TO RELATED APPLICATIONS

This application claims the benefit of U.S. Ser. No. 61/490,938, filed May 27, 2011, the contents of which is incorporated by reference in its entirety.

### BACKGROUND OF THE INVENTION

The present invention relates to lenses, which may include, for example, ophthalmic lenses such as spectacle lenses, contact lenses and intra-ocular lenses. More specifically, the present invention relates to ophthalmic lenses including a plurality of dynamic regions activated by a deformable surface.

Ophthalmic lenses are fabricated to individual prescriptions and frames, requiring a “one of” customized manufacturing process that is time consuming and expensive. Recent developments of adjustable power lens technology such as liquid filled lenses, or lenses with dynamic, switchable add power enable consumers to adjust the lens power over a limited range in single vision, multifocal or progressive addition lenses designed to provide correction at far and near distances.

The standard tools for correcting presbyopia are reading glasses, multifocal ophthalmic lenses, and monocular fit contact lenses. Reading glasses have a single optical power for correcting near distance focusing problems. A multifocal lens is a lens that has more than one focal length (i.e., optical power) for correcting focusing problems across a range of distances. Multifocal ophthalmic lenses work by means of a division of the lens's area into regions of different optical powers. Multifocal lenses may be comprised of continuous surfaces that create continuous optical power as in a Progressive Addition Lens (PAL). Alternatively, multifocal lenses may be comprised of discontinuous surfaces that create discontinuous optical power as in bifocals or trifocals.

Electronic ophthalmic lenses for presbyopic wearers (those over the age of 40 years who have difficulty seeing clearly at near distances of 14-18 inches and/or intermediate distances of 18+ inches to 36 inches) have been taught for contact lenses, intra ocular lenses and spectacle lenses.

The emerging technologies that involve adjustable power lens technology, such as liquid filled lenses, or lenses with dynamic, switchable add power, have significant limitations. For example, fluid filled lenses require a reservoir of additional fluid that has to be pumped into the lens in order to effect change of power. The presence of a reservoir of additional fluid causes the eyeglass to become bulky and fragile, since any rupture of the reservoir makes the lens inoperable, and may cause spill of a chemical, potentially harming the wearer.

In practice, the adjustable range of power in fluid filled lenses is less than 2.00 Diopters, particularly if the optical power is designed to be provided full field, rather than over a relatively narrow corridor or viewing zone centered around the optical center of the lens.

Similarly, the range of adjustability of electro-active, switchable optical elements is effectively less than 1.50 diopters, even when it is provided over a relatively small segment situated within the overall optic.

In addition, in many cases fluid lenses and also electro-active lenses involve a static lens component which is in optical communication with the dynamic fluid lens or the dynamic electro-active lens.

Present day static eyeglass lenses which have evolved over the last 600 plus years must be ground and polished to the prescription of the wearer. Following this they must be edged and mounted into an eyeglass frame. The customization process which exists today with the fabrication of eyeglasses adds substantial costs, and delays the consumer from receiving his or her eyeglasses.

The following discloses an inventive programmable lens capable of creating optical power covering most, if not all optical power prescriptions and whereby said inventive lens can be dynamically changed in optical power.

### BRIEF SUMMARY OF THE INVENTION

Aspects of the present invention may relate generally to ophthalmic, or other, lenses including a plurality of adjustable regions, in which the adjustment of optical power is provided by active deformation of a lens surface.

According to first aspects of the invention, an ophthalmic lens may be provided comprising a deformable layer, a membrane disposed opposite the deformable layer and a patterned electrode. Embodiments may include at least two regions of adjustable optical power. At least part of the membrane may be configured to move axially along an optical path of the lens, and a surface of the deformable layer may be configured to at least one of expand and contract based on movement of the at least part of the membrane along the optical path of the lens.

In embodiments, adjustment in optical power may be provided by using a deformable optically transparent gel. The deformation of the gel may be driven, for example, by a transparent membrane that functions like a piston. The membrane may be driven by piezoelectric, or similar forces.

In embodiments, the at least two regions of adjustable optical power may include separate regions corresponding to individually addressable portions of the patterned electrode.

Embodiments may further include a rigid optical element. In embodiments, the deformable layer may be disposed between the membrane and the rigid optical element.

In embodiments, the rigid optical element may include a raised edge that at least partially surrounds a circumference of the deformable layer.

In embodiments, the rigid optical element may include a raised edge that substantially surrounds a circumference of the deformable layer.

In embodiments, the rigid optical element may be disposed on an anterior side of the lens, and the membrane may be disposed on a posterior side of the lens.

In embodiments, the rigid optical element may be disposed on a posterior side of the lens, and the membrane may be disposed on an anterior side of the lens.

In embodiments, the rigid optical element may provide an optical power of at least one of −7.00 D, −2.00 D, +2.00 D, +3.50 D, +6.50 D, +8.50 D to the lens.

In embodiments, the rigid optical element may be aspherized. In embodiments, the rigid optical element may provide zero optical power to the lens.

In embodiments, the deformable layer may be bonded to at least one of the rigid optical element and the membrane.

In embodiments, an optical power of the lens may be dynamic and/or tunable.

In embodiments, the axial movement of the membrane may change a topography of the lens.

In embodiments, the axial movement of the membrane may change a posterior surface topography of the lens

In embodiments, the deformable layer may be configured to adjust an optical power of the lens via physical deformation of the deformable layer.

In embodiments, the deformable layer may be configured to adjust a base power of the lens in a range of approximately ±5 diopter via physical deformation of the deformable layer.

In embodiments, the membrane may beconfigured to be driven by piezoelectric forces.

In embodiments, the membrane may include PVDF (Polyvinyledene difluoride).

In embodiments, the membrane may be configured to form a sag profile that departs from a resting position by up to approximately 200 microns.

In embodiments, the membrane may be configured to deflect in both directions along the optical path of the lens.

In embodiments, the deformable layer may include an optically transparent gel.

In embodiments, the gel may have a refractive index that is different from a refractive index of another layer of the lens.

In embodiments, wherein the gel may include cross linked silicone elastomers.

In embodiments, the deformable layer may have a thickness in the range 1.0 mm to 10.0 mm.

In embodiments, the patterned electrode may be a transparent electrode on at least one surface of the membrane.

In embodiments, the lens may be configured to form an aspheric power contour upon actuation of the transparent electrode.

Embodiments may include transparent electrodes on each of a posterior surface and an anterior surface of the membrane.

In embodiments, the patterned electrode may be disposed on at least one surface of the membrane.

In embodiments, the patterned electrode may include a grid corresponding to a plurality of individually addressable pixels.

In embodiments, the lens may be configured to correct for non-conventional refractive error via selective movement of portions of the membrane.

In embodiments, the rigid optical element may be configured to provide a toric correction (astigmatic optical power).

In embodiments, the membrane may be configured to provide a toric correction (astigmatic optical power).

In embodiments, the lens may be configured to change optical power to correct for far, intermediate, and near vision correction needs of a wearer.

Embodiments may further include a controller configured to adjust the membrane.

In embodiments, the controller may be programmable to provide a set of predetermined voltages to the membrane for correcting for far, intermediate, and near vision correction needs of a wearer.

In embodiments, the controller may be remotely programmable, and allows the lens to be reconfigured based on needs of the wearer.

In embodiments, the lens is at least one of an a spectacle lens, contact lenses and intra-ocular lenses, a camera lens, a lens for a medical device, or a lens for an optical scanner.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention claimed. The detailed description and the specific examples, however, indicate only preferred embodiments of the invention. Various changes and modifications within the spirit and scope of the invention will become apparent to those skilled in the art from this detailed description.

### BRIEF DESCRIPTION OF THE DRAWINGS

Aspects and features of the invention will be understood and appreciated more fully from the following detailed description in conjunction with the figures, which are not to scale, in which like reference numerals indicate corresponding, analogous or similar elements.

FIG. 1 shows a schematic cross sectional view of a lens structure according to aspects of the invention.

FIG. 2 shows a schematic plan view of a lens structure depicting contours of optical power according to aspects of the invention.

FIG. 3 shows a power profile of a lens according to aspects of the invention.

FIG. 4 shows further details of an exemplary deformable membrane according to aspects of the invention.

FIG. 5 shows further details of a patterned electrode according to aspects of the invention.

FIG. 6 shows another embodiment of a lens including a plurality layers according to further aspects of the invention.

FIG. 7 shows an example of a spectacle frame including a controller according to further aspects of the invention.

FIG. 8 shows another example of a spectacle frame, and lenses with embedded ASIC's, according to further aspects of the invention.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a layer” is a reference to one or more layers and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals reference similar parts throughout the several views of the drawings.

The following preferred embodiments may be described in the context of exemplary active ophthalmic lens devices for ease of description and understanding. However, the invention is not limited to the specifically described devices and methods, and may be adapted to various assemblies without departing from the overall scope of the invention. For example, devices and related methods including concepts described herein may be used for other lenses and optical systems, and other apparatus with dynamic lenses using physical deformation of a lens surface and/or internal component.

As used herein, an electro-active element refers to a device with an optical property that is alterable by the application of electrical energy, whereas an active element more broadly refers to a device with an optical property that is alterable by various means including the application of electrical energy. According to aspects of the invention, active elements, including electro-active elements, may be used in exemplary lenses to provide a plurality of regions with adjustable optical power, such as by using an electrically deformable layer (or “membrane”) and a patterned electrode with separately addressable regions.

In general, an alterable optical property may be, for example, optical power, focal length, diffraction efficiency, depth of field, optical transmittance, tinting, opacity, refractive index, chromatic dispersion, or a combination thereof. However, in the context of the present subject matter, the alterable optical property may more particularly refer to, for example, optical power, focal length, depth of field or a combination thereof.

An electro-active element may be constructed from two substrates and a deformable gel disposed between the two substrates. Typically, one of the substrates will be substantially rigid and the other substrate is deformable based on the application of electricity or other forces. Alternatively, both of the substrates may be deformable, individually and/or in synchronization. One or both of the substrates may be shaped and sized to ensure that the gel material is contained within the substrates. The gel, and/or a gel container, may be bonded to one or both of the substrates. One or more transparent electrodes may be disposed on a surface of the substrates. One or more of the electrodes may be patterned to substantially correspond to active regions of the electro-active element.

The electro-active element may include a power supply operably connected to a controller. The controller may be operably connected to the electrodes by way of electrical connections to apply one or more voltages to each of the electrodes.

When electrical energy is applied to a deformable membrane by way of the electrodes, the optical power of the lens may be altered. For example, when electrical energy is applied to a deformable layer by way of the electrodes, the topography of a lens surface may be altered, thereby changing the optical power of the lens.

The active element may be embedded within or attached to a surface of an ophthalmic lens to form an active lens. Alternatively, the active element may be embedded within or attached to a surface of an optic which provides substantially no optical power to form an active optic. In such a case, the active element may be in optical communication with an ophthalmic lens, but separated or spaced apart from or not integral with the ophthalmic lens. The ophthalmic lens may be an optical substrate or a lens.

A “lens” is any device or portion of a device that causes light to converge or diverge (i.e., a lens is capable of focusing light). A lens may be refractive or diffractive, or a combination thereof. A lens may be concave, convex, or planar on one or both surfaces. A lens may be spherical, cylindrical, prismatic, or a combination thereof. A lens may be made of optical glass, plastic, thermoplastic resins, thermoset resins, a composite of glass and resin, or a composite of different optical grade resins or plastics. It should be pointed out that within the optical industry a device can be referred to as a lens even if it has zero optical power (known as plano or no optical power). However, in this case, the lens is usually referred to as a “plano lens.” A lens may be either conventional or non-conventional. A conventional lens corrects for conventional errors of the eye including lower order aberrations such as myopia, hyperopia, presbyopia, and regular astigmatism. A non-conventional lens corrects for non-conventional errors of the eye including higher order aberrations that can be caused by ocular layer irregularities or abnormalities. The lens may be a single focus lens or a multifocal lens such as a Progressive Addition Lens or a bifocal or trifocal lens. Contrastingly, an “optic”, as used herein, has substantially no optical power and is not capable of focusing light (either by refraction or diffraction). The term “refractive error” may refer to either conventional or non-conventional errors of the eye. It should be noted that redirecting light is not correcting a refractive error of the eye. Therefore, redirecting light to a healthy portion of the retina, for example, is not correcting a refractive error of the eye.

The active element may be located in the entire viewing area of the active lens or optic or in just a portion thereof. The active element may be located near the top, middle or bottom portion of the lens or optic. It should also be noted that the active element may be capable of focusing light on its own and does not need to be combined with an optical substrate or lens.

As used herein, various active regions may be referred to as a first region, a second region, a third region, etc., with or without relation to one another. For example, a first region and second region may be disposed in separate areas of a lens, a first region may be encompassed by a second region (which may be annular), etc. The regions may have at least one optical characteristic that is different among the regions. For example, a first region may have a different optical transmission, refractive index, or optical path length than the second region, based on features such as an optical power of a corresponding region of a rigid lens portion and/or a variation in the characteristics of the active element in the first and second regions.

The invention disclosed herein relates to various embodiments of active lenses including ophthalmic lenses. Ophthalmic lens as defined herein refer to spectacle eyeglass lenses, or any similar lens that focuses, transmits, directs, and or refracts light onto the retina of the user/wearer's eye. When used as a spectacle lens, a tilt switch or similar sensor connected to an ASIC or micro controller may cause the spectacle lens to change its optical power.

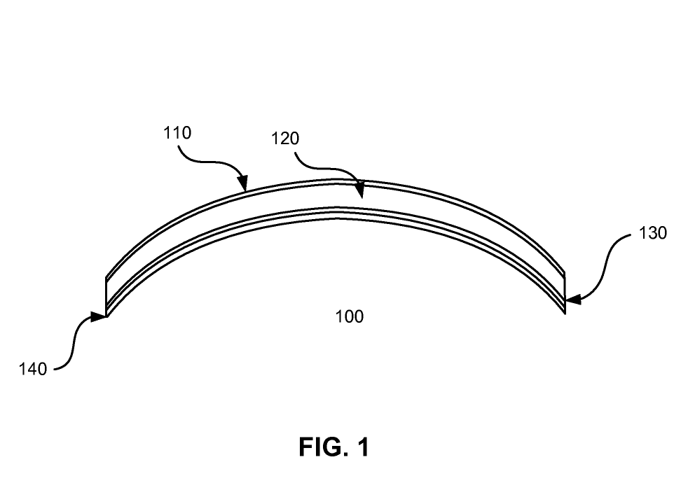

As shown in FIG. 1, according to aspects of the invention, an ophthalmic lens 100 may include a rigid layer 110, a deformable layer 120, and a deformable membrane 130 disposed opposite the deformable layer 120. In this embodiment, the membrane 130 is disposed toward the rear of the lens (i.e. closer to the patient's eye). However, other configurations are possible, including disposing the deformable membrane on an anterior side of the lens, and/or when both the anterior and posterior sides of the lens include a deformable membrane.

The deformable layer 120 may include an optically transparent gel. In embodiments, the gel may have a refractive index that is different from a refractive index of another layer and/or element of the lens, such as an optical element of the rigid layer 110 and/or the membrane 130. In embodiments, the gel may include cross linked silicone elastomers. The deformable layer 120 may have a thickness in the range of, for example, 1.0 mm to 10.0 mm.

The deformable layer 120 may be bonded to the rigid layer 110 and/or the membrane 130. Preferably, the deformable layer, e.g. the deformable gel or a gel container, is bonded to both the rigid layer 110 and also to the membrane 130 to enhance the selective deformation of the deformable layer 120 based on movement of the membrane 130.

An axial movement of the membrane 130 may change a topography of the lens 100, e.g. the axial movement of the membrane 130 may change a posterior surface topography of the lens 100 and thereby change an optical power provided by the lens 100. The membrane 130 may be configured to be driven, for example, by piezoelectric or other forces. The membrane 130 may be made of a material having a high piezoelectric coefficient, such as, for example, PVDF (Polyvinyledene difluoride). As discussed further below, different regions of the rigid layer 110 and/or membrane 130 may provide different regions of the lens with variable optical power. The lens 100 may be configured, for example, to correct for non-conventional refractive error via selective movement of portions of the membrane 130.

The membrane 130 may be configured to form a sag profile that departs from a resting position by up to, for example, approximately 200 microns.

In embodiments, the membrane 130 may be configured to deflect in both directions along the optical path of the lens.

The deformable layer 120 may be configured to adjust a base power of the lens in a range of, for example, approximately ±5 diopter via physical deformation of the deformable layer 120 and membrane 130.

The rigid layer 110 and/or the membrane 130 may include optical elements, or may be configured to provide zero optical power in all of part of the layer. The rigid layer may include one or more optical elements configured to provide an optical power including one or more of −7.00 D, −2.00 D, +2.00 D, +3.50 D, +6.50 D, +8.50 D to the lens.

The rigid layer 110 may have a refractive index that is preferably equal or close to that of that of a gel contained in the deformable layer 120. While this is preferred it is not mandated. The refractive index of the rigid layer 110 and the gel is preferably within 1.50 to 1.80, preferably 1.60 and 1.70. The rigid layer 110 may be made of a high index plastic material, such as polycarbonate of bisphenol A, or a copolymer of thioacrylates, methacrylates, amides or ureas, or mineral glass of refractive index in the range of 1.50 to 1.80.

The rigid layer 110 may have a front curvature ranging from 0.5 D to 10.0 D (1000 mm to 50 mm in radius of curvature). Alternatively, the rigid layer may include a range of front curvatures and optical powers, including a range of base curves covering a prescription range of, for example, −10.00 D to +10.00 D. This may include, for example, between 5-15 front curves.

The rigid layer 110 may include an optical element (not shown) to provide a toric correction (astigmatic optical power). It should also be noted that the membrane 130 may be configured to provide a toric correction (astigmatic optical power). The rigid layer 110 may be aspherized. In other embodiments, the lens may be configured such that the rigid layer 110 provides zero optical power to the lens.

The rigid layer 110 may include a raised edge that at least partially surrounds a circumference of the deformable layer 120, or that substantially surrounds a circumference of the deformable layer

As mentioned above, other configurations are also possible, such as those in which the rigid layer is disposed on a posterior side of the lens, and the membrane is disposed on an anterior side of the lens.

The membrane 130 may be coated on one or more surfaces with a layer of indium tin oxide (ITO) or any other substantially transparent electrically conductive material that can function as an electrode. For example, as also shown in FIG. 1, the lens 100 includes a transparent electrode 140, which may be a patterned electrode. Electrode 140 is disposed on the posterior side of the membrane 130. However, it should be noted that transparent electrodes may be disposed on each of the posterior surface and the anterior surface of the membrane 130. Typically, one of the electrodes is that of a patterned electrode. The pattern can be that of any configuration or that of a grid comprising individually addressable pixels. The pixels can be of any distance apart but preferably approximately 1 mm apart.

The electrode 140 may include a plurality of separately addressable regions, such as concentric circles, ellipsoids or annuluses, non-overlapping regions, pixels, etc. For example, the electrode 140 may include a grid corresponding to a plurality of individually addressable pixels.

The lens 100 may include at least two regions of adjustable optical power, which may correspond to individually addressable portions of the electrode 140. That is, the electrode 140 (or electrodes, i.e., one or both surfaces) is preferably patterned, so that each segment is separately addressable when connected to a electrical bus by means of switchable circuit. As discussed further below, the switching points may be driven by a miniaturized logic controller, which may also reside on the edge of the rigid layer 110 or a recess between the edge of the rigid layer and the frame.

Upon application of an electrical potential to a particular segment of the electrode 140, the area of the membrane 130 in contact with that electrode segment changes it sag thus changing the optical power of the lens 100 by changing the back surface topography/curvature of the lens. For example, the membrane 130 deforms the gel in contact with it, causing either compression or extension, depending on the direction of the electrical potential.

Application of electric potential across a set of individual segments within the electrode pattern can be used to develop any sag profile in the gel, within the limits of the magnitude of piezoelectric response of the membrane and also the limit of elastic deformation of the gel underneath the membrane.

Commercially available membrane materials can provide a piezoelectric response of 5% or less, i.e., 50 microns per millimeter. Therefore, segment that is 20 mm in any dimension in the xy plane can be driven over a sag range of 100 microns. This change in sag is well within the limit of deformability of commercially available gels, such as but not limited to cross linked silicone elastomers. The change in sag of the gel is related to change in optical power, depending on the refractive index of the gel and the curvature of the gel's front surface, which is contiguous with the posterior surface of the rigid front optical element to which it is bonded.

By way of further example, a change in optical power of 1.000 is provided by a gel of refractive index of 1.52, for a change in sag of 100 microns over a 20 mm segment with a front curvature of 5.00. The change in optical power will be proportional to the refractive index of the gel in the ratio of (n1−1)/(n2−1), in which n1 is 1.52 and n2 is the refractive index of the gel. Thus a gel of refractive index 1.60 will provide a power change of 1.150 for a change in sag of 100 microns over a linear dimension of 20.0 mm.

It is thus possible to create any sag profile within the limits of piezoelectric response of the membrane and the elasticity of response of the gel. The front rigid front optical element may be without any net optical power, or it may provide an optical power. In certain embodiments of the invention the front rigid optical element can provide one or more of plus optical power, minus optical power, astigmatic optical power, additive plus optical power such as that of a progressive addition lens.

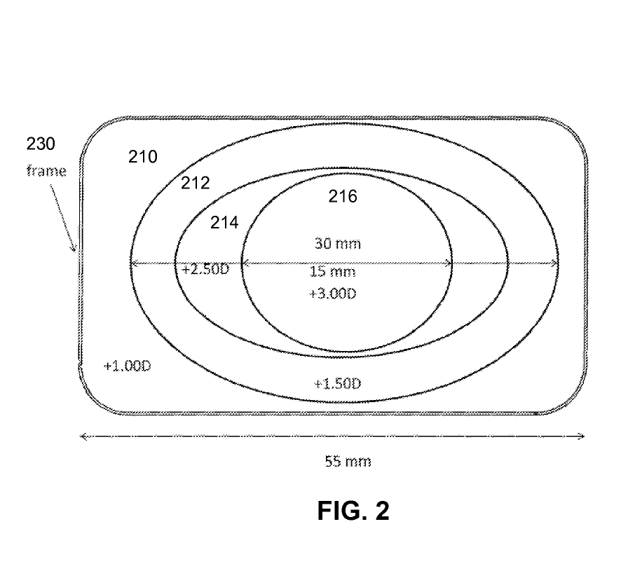

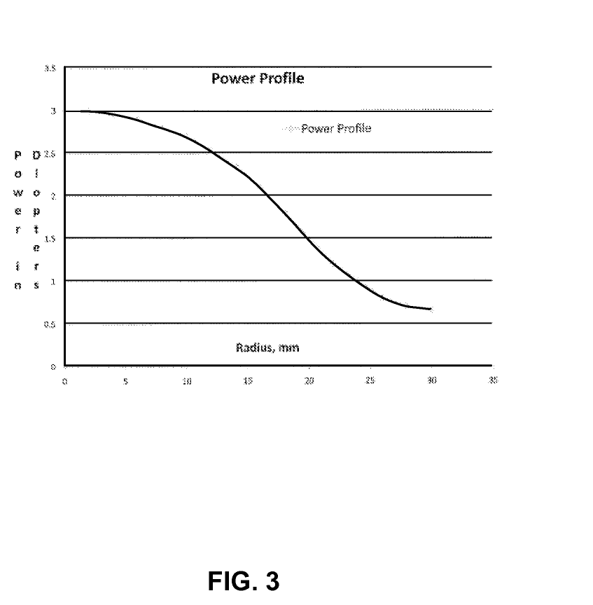

The curvature of the front rigid optical element is dependant on the range of ophthalmic corrections to be provided. The profile of dynamic power increment may be circularly symmetric, or it may have a four fold symmetry creating an aspheric optic, as shown in FIG. 2-3.

The optical power of the rigid element will depend on its front curvature as shown in Table 1. In this regard, embodiments of the invention may include hybrid lenses whereby some or all of the add power is found on the front rigid optical element.

TABLE 1

| Lens Range of Optical | Base Curve of Front | Power of Front Rigid |
|---|---|---|
| Power | Rigid Optical Element | Optical Element |
| −10.00 D to −5.00 D | 0.50 D | −7.00 D |
| −4.750 to 0.0 D | 1.50 D | −2.00 D |
| +0.25 D to 2.00 DD | 4.00 D | +2.00 D |
| +2.225 D to 5.0 D | 5.50 D | +3.50 D |
| +5.25 D to 7.50 D | 6.50 D | +6.50 D |
| +7.75 D to 10.0 D | 8.00 D | +8.50 D |

The above is by way of example only to show how to divide up the optical power from −10.000 to +10.000 by base curve by major optical component, or said another way to show the relationships of base curve, rigid optical element, and inventive lens optical power. Note multiple base curves allow for the possibility of creating a range of inventive optical powers from +10.000 to −10.000. Also while add power is not shown, the inventive lens allows for covering all add powers from +0.750 to +3.500. In these examples, the front optical element is preferably aspherized.

Embodiments may include at least two regions of adjustable optical power, such as regions 210, 212, 214 and 216 shown in FIG. 2, which include different optical power and may be separately addressable via patterned electrode. Different segments of the patterned electrode may be programmable and/or configured to provide different electrical power to different regions of the deformable membrane. At least part of the membrane may be configured to move axially along an optical path of the lens, and a surface of the deformable layer may be configured to at least one of expand and contract based on movement of the at least part of the membrane along the optical path of the lens. In embodiments, the membrane topography may be alterable to provide for correcting presbyopia either fully or partially.

Thus, embodiments such as shown in FIGS. 1 and 2 may include a plurality of active regions. It should be noted that, in certain embodiments, the active regions may not cover an entire surface of the lens and may be limited to a certain portion of the lens. For example, the region 210 shown in FIG. 2, or other portions of the lens, may not include an active element. Otherwise, each of the plurality of active regions may provide increased optical add power when an electrical potential is applied by altering the local topography and/or thickness of the lens. The application of an electrical potential can be directed to each of the active regions, a group of these regions, or all of the regions simultaneously. The plurality of active regions as shown in FIG. 2 are located as rings of such regions located around a single central active region. The optical power of these regions when activated can be within the range of +0.75 D to +3.50 D, and even more preferably within the range of +1.00 D to +3.00 D.

In embodiments, exemplary lenses, such as shown in FIGS. 1, 2, 4 and 6, may contain a plurality of dynamic optical power regions within an add power region. The term dynamic means the optic is capable of changeable optical power as opposed to being a fixed static optical power. The add power region is the region of the lens that dynamically increases plus optical power over and beyond the distance optical power. This change can be in steps of optical power or by way of continuous optical power.

It should be pointed out that, according to embodiments, given the size and arrangement of each region and its corresponding dynamic optical power, the depth of focus may be increased as the optical power is dynamically increased.

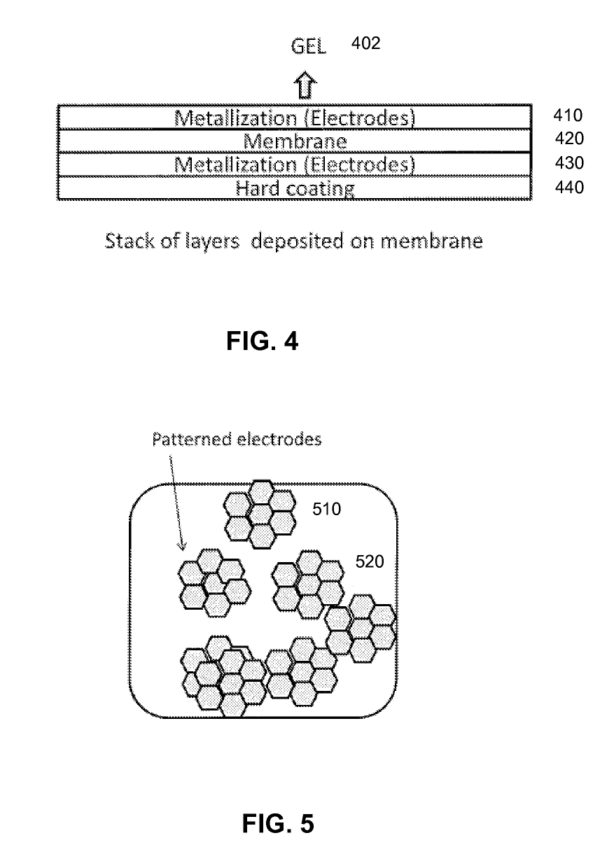

Additional details of an exemplary deformable membrane assembly are shown in FIG. 4. As shown in FIG. 4, a deformable membrane assembly may include a first electrode 410 disposed toward, or in contact with, a gel layer 402. A deformable membrane 420 may be disposed between the first electrode 410 and a second electrode 430. One or more coatings, such as a hard coating 440, may be deposited on an exterior surface of the deformable membrane assembly. Activation of portions of the electrode 410 or 430 may force movement of the deformable membrane 420 toward, or away, from the gel 402, which alters the surface topography and local thickness of the lens.

As mentioned previously, in the present subject matter, electrodes, such as electrodes 410 or 430 may be patterned to form particular regions of the lens system. An example of such patterning is shown in FIG. 5, which shows a number of electrodes configured in clusters 510, 520, of pixels. In the case of concentrically arranged pixel elements such as shown in FIG. 5, it is possible to create micro-lenses in each of the areas by individually addressing the pixels. Other configurations are also possible, such as concentric rings, full-field pixel arrangements, etc.

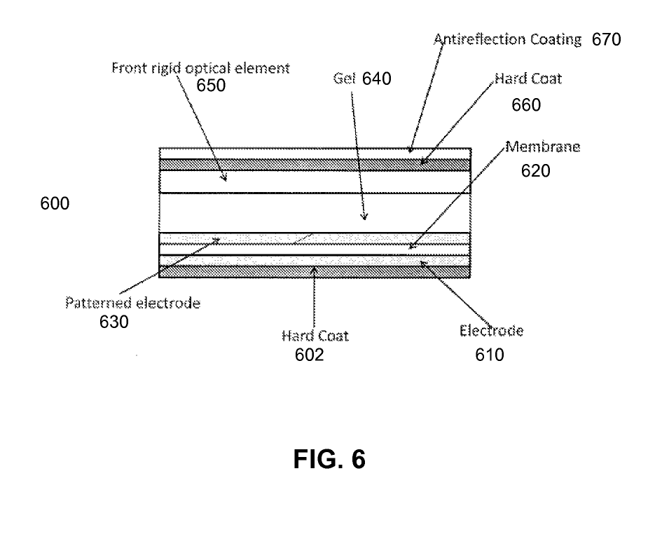

Further details regarding the layers of lenses according to aspects of the invention are shown in FIG. 6. As with some of the other embodiments discussed herein, the lens shown in FIG. 6 includes a membrane 620 between electrodes 610 and 630. Either or both of electrodes 610 and 630 may be pattered appropriately. A gel layer 640 may be bonded on a posterior side to the electrode 630 and/or the membrane 620. For example, in circumstances where the electrode 630 is patterned, the gel layer 640 may be bonded to the electrode 630 where it exists, and to the membrane 620 in locations where the electrode 630 does not exist. Gel layer 640 may be bonded, on an opposite anterior side, to a rigid optical element 650. The inventive lens can be hard coated on either exterior surface if desired. As shown in FIG. 6, an inventive lens may also include a hard coat 660 and/or an antireflection coating 670 on the front surface of front rigid optical element 650. The antireflection coating 670 can comprise a smudge free coating (not shown) on its outer surface.

As noted previously, while the lens shown in FIG. 6 depicts the rigid optical element on the front and the electro-formable membrane on the back having the electrode layers adjacent thereof, in certain other inventive embodiments the rigid optical element is on the back and the electro-formable membrane on the front having the electrode layers adjacent.

A controller may also be provided (internal or external to lens 100) that is configured to adjust the membrane 130. The controller may be remotely programmable, and allow the lens to be reconfigured based on needs of the wearer. An optical power of the lens 100 may be dynamic and/or tunable, as discussed above.

While it is understood that the lenses could be controlled directly by the ASIC, remote programming will be preferred because the gel layer may have complex shapes and curvatures in areas between electrodes, for a given set of applied voltages. The voltages may therefore need to be fined tuned across the lens to deal with the “cross talk” between electrodes. To handle this directly with the ASIC would place undue demands on its computational requirement. It would be a preferred embodiment to therefore optimize the power to a set predetermine voltages remotely with a more complex controller than the ASIC to determine the precise voltages to be applied to each electrode for a given correction mode (far, intermediate, or near). In this manner, the functionality of the ASIC can be limited to monitoring the sensors, setting the voltages for each electrode based on a programmed look up tables for various corrections, drive the lenses, or other low computationally intensive tasks.

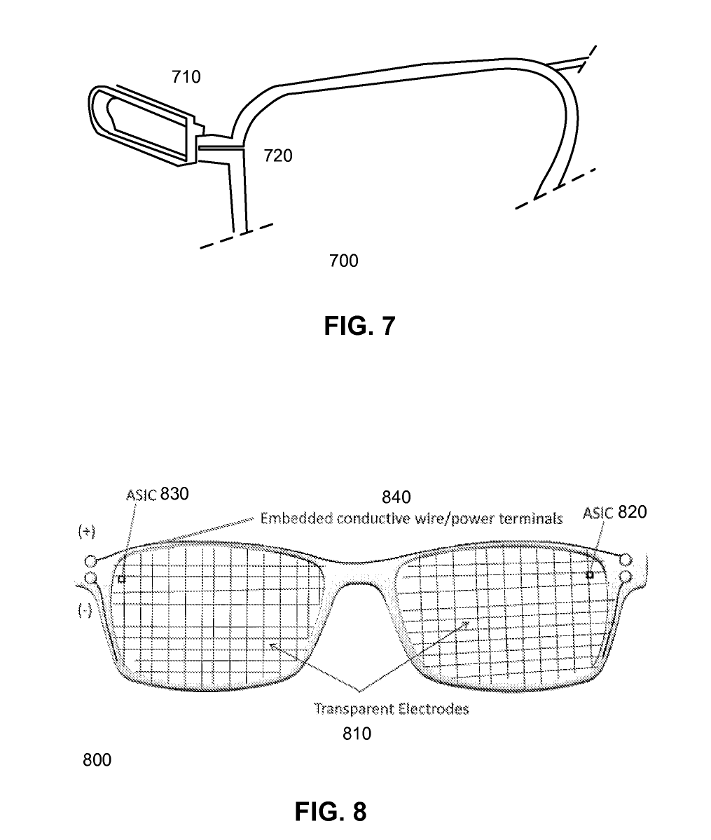

As shown in FIG. 7, an exemplary frame may be used for eyeglasses including a pre-shaped electronic filler lens having a predetermined base curve, and that is both adjustable and dynamic as described herein. According to aspects of the invention, an eyeglass frame, such as frame 700 shown in FIG. 7, may include electronics, such as a controller and power source, disposed in housing 710, that enable, activate, provide sensing, and direction to the electronic lens or lenses housed therein, such as via connections within hinge 720.

Alternatively, lenses may include one or more of the electronic controllers described herein. For example, as shown in FIG. 8, eyeglasses 800 may include ASICs 820, 830, located on or within the lenses of the eyeglasses. As shown in FIG. 8, the lenses may also include transparent electrodes 810, which are patterned in a suitable configuration, as well as power terminals 840 for receiving power from a power source, such as batteries in other parts of the frame (not shown).

The lens may further include a battery, such as an inductive thin-film battery, a power management system and/or sensors, which may be, for example, photosensors. Such components may be disposed completely, or partly, within a peripheral region of the lens, such as in region 210 shown in FIG. 2.

According to aspects of the invention, eyeglasses may be configured to be programmed immediately following the completion of an eye examination or simultaneous with the eye examination of the wearer. In embodiments, the eyeglasses may be programmed remotely or directly, e.g. via various electronic links suitable for exchanging data known to those of skill in the art.

The lens, such as lens 100, may be configured to change optical power to correct for far, intermediate, and near vision correction needs of a wearer. For example, the controller may be programmable to provide a set of predetermined voltages to the membrane for correcting for far, intermediate, and near vision correction needs of a wearer. In embodiments, the lens may be configured to form an aspheric power contour upon actuation of the transparent electrode.

The remote programmer may also be configured to not only set the drive voltages but to also fine tune the Rx in the range of desired corrections using the glasses as an electro-active as part of an electro-active eye exam for setting correction for far, near, and intermediate vision. This may also allow for more flexibility in the tolerances in layer thickness, and other properties of the lenses thus keeping manufacturing cost low.

According to aspects of the inventions, lenses may be configured to correct for myopia, hyperopia, astigmatism, or a combination of these. As will be appreciated, the inventive lens can also be dynamically altered between two or more prescriptions.

According to further aspects of the invention, inventive lenses and/or frames may include a sensor such as, by way of example only, a microaccelerometer, tilt switch, micro gyroscope, range finder that provides feed back to the controller thus providing an electrical signal or electrical signals that results in a change of the profile of the electrical potential thus causing the optical power of the lens to dynamically change.

Although described in the context of a spectacle lens, aspects of the lens 100 may also find applicability in the contexts of other lenses, such as contact lenses, intra-ocular lenses, a camera lens, a lens for a medical device, a lens for an optical scanner, etc.

It should also be noted that the lens 100, and particularly the rigid layer 110, may include various alternative and/or additional features, such as, for example, one or more active regions including liquid crystal, electro-chromic or other materials, a plurality of dynamic micro-lenses or micro-prismatic apertures, etc.

In certain cases, the active element (e.g. the deformable layer and/or membrane) may cover the majority of the optical surface of the ophthalmic host lens, e.g. the rigid layer. In other embodiments, the active element may cover less than the majority of the optical surface of the ophthalmic host lens. This could be, for example, for the use of the invention with certain types of multi-focal spectacle lenses and/or gaming or entertainment spectacles or eyewear.

In embodiments where a liquid crystal element may be combined with the inventive lens, e.g. to provide an electro-chromic or other effect, such liquid crystals may include, by way of example only, nematic, cholesteric. The liquid crystal can also be made to be dichroic by formulating a dichroic dye within the liquid crystal such that it will turn dark (change light absorption) when switched. In many cases, a single layer of cholesteric liquid crystal may be used.

According to embodiments of the invention, two electrodes made of transparent electrodes by way of example only, such as indium tin oxide, may be provided, preferably on either side of a deformable membrane. Other positioning of the electrodes is also possible, e.g. one electrode on the inside layers of opposing substrates, one electrode being located on the innermost surface of one substrate and the outermost surface of another substrate, or both electrodes being located on the outermost surface of both substrates. The invention also contemplates these substrates being comprised of, by way of example only, glass, plastic or a combination of both.

A self contained sealed electro-active module may be provided in various of the embodiments, and may generally comprise the active deformable membrane assembly with, or without, the a deformable layer assembly, e.g. a gel layer or packet. The active deformable membrane assembly may include the necessary electrodes and deformable membrane, as well as connectors for connecting to a controller and/or power supply. In embodiments the self contained sealed electro-active module may be configured for easy attachment to a fixed optic, such as the fixed layer described herein.

When the inventive embodiment is that of a spectacle lens the sensing is that of, by way of example only, a range finder, micro-accelerometer, tilt switch, micro-gyroscope, capacitor touch/swipe switch. Any one or all of these sensors can be built into the inventive ophthalmic host lens or that of the eyeglass frame that houses the inventive dynamic spectacle lens.

It should be pointed out that all measurements, dimensions, optical powers, shapes, figures, illustrations, provided herein by way of example and are not intended to be self limiting.

As described above, various exemplary lenses may include embedded sensors. The sensor may be, for example, a range finder for detecting a distance to which a user is trying to focus. The sensor may be light-sensitive cell for detecting light that is ambient and/or incident to the lens or optic. The sensor may include, for example, one or more of the following devices: a photo-detector, a photovoltaic or UV sensitive photo cell, a tilt switch, a light sensor, a passive range-finding device, a time-of-flight range finding device, an eye tracker, a view detector which detects where a user may be viewing, an accelerometer, a proximity switch, a physical switch, a manual override control, a capacitive switch which switches when a user touches the nose bridge of a pair of spectacles, a pupil diameter detector, a bio-feed back device connected to an ocular muscle or nerve, or the like. The sensor may also include one or more micro electro mechanical system (MEMS) gyroscopes adapted for detecting a tilt of the user's head or encyclorotation of the user's eye.

The sensor may be operably connected to a lens controller. The sensor may detect sensory information and send a signal to the controller which triggers the activation and/or deactivation of one or more dynamic components of the lens or optic.

The sensor, by way of example only, may detect the distance to which one is focusing. The sensor may include two or more photo-detector arrays with a focusing lens placed over each array. Each focusing lens may have a focal length appropriate for a specific distance from the user's eye. For example, three photo-detector arrays may be used, the first one having a focusing lens that properly focuses for near distance, the second one having a focusing lens that properly focuses for intermediate distance, and the third one having a focusing lens that properly focuses for far distance. A sum of differences algorithm may be used to determine which array has the highest contrast ratio (and thus provides the best focus). The array with the highest contrast ratio may thus be used to determine the distance from a user to an object the user is focusing on.

Some configurations may allow for the sensor and/or controller to be overridden by a manually operated remote switch. The remote switch may send a signal by means of wireless communication, acoustic communication, vibration communication, or light communication such as, by way of example only, infrared. By way of example only, should the sensor sense a dark room, such as a restaurant having dim lighting, the controller may cause changes to the lens that impact the user's ability to perform near distance tasks, such as reading a menu. The user could remotely control the lens or optic to increase the depth of field and enhance the user's ability to read the menu. When the near distance task has completed, the user may remotely allow the sensor and controller to act automatically thereby allowing the user to see best in the dim restaurant with regard to non-near distance tasks.

While illustrative and presently preferred embodiments of the invention have been described in detail herein, it is to be understood that the inventive concepts may be otherwise variously embodied and employed, and that the appended claims are intended to be construed to include such variations, except as limited by the prior art.

1. An adaptable ophthalmic lens, comprising:
a deformable layer;
a membrane disposed opposite the deformable layer;
a patterned electrode; and
at least two regions of adjustable optical power,
wherein, at least part of the membrane is configured to move axially along an optical path of the lens, and
a surface of the deformable layer is configured to at least one of expand and contract based on movement of the at least part of the membrane along the optical path of the lens.

2. The lens of claim 1, wherein the at least two regions of adjustable optical power include separate regions corresponding to individually addressable portions of the patterned electrode.

3. The lens of claim 1, further comprising a rigid optical element, wherein the deformable layer is disposed between the membrane and the rigid optical element.

4. The lens of claim 3, wherein the deformable layer is bonded to at least one of the rigid optical element and the membrane.

5. The lens of claim 1, wherein an optical power of the lens is dynamic.

6. (canceled)

7. The lens of claim 1, wherein the axial movement of the membrane changes a topography of the lens.

8. (canceled)

9. The lens of claim 1, wherein the deformable layer is configured to adjust an optical power of the lens via physical deformation of the deformable layer.

10. (canceled)

11. The lens of claim 1, wherein the membrane is configured to be driven by piezoelectric forces.

12-14. (canceled)

15. The lens of claim 1, wherein the deformable layer includes an optically transparent gel.

16. (canceled)

17. (canceled)

18. The lens of claim 1, wherein the deformable layer has a thickness in the range 1.0 mm to 10.0 mm.

19-22. (canceled)

23. The lens of claim 3, wherein the rigid optical element provides an optical power of at least one of −7.00 D, −2.00 D, +2.00 D, +3.50 D, +6.50 D, +8.50 D to the lens.

24. The lens of claim 3, wherein the rigid optical element is aspherized.

25. The lens of claim 3, wherein the rigid optical element provides zero optical power to the lens.

26. The lens of claim 1, wherein the patterned electrode is a transparent electrode disposed on at least one surface of the membrane.

27. The lens of claim 26, wherein the lens is configured to form an aspheric power contour upon actuation of the transparent electrode.

28. The lens of claim 1, further comprising transparent electrodes on each of a posterior surface and an anterior surface of the membrane.

29. The lens of claim 1, wherein the patterned electrode is disposed on at least one surface of the membrane.

30. The lens of claim 1, wherein the patterned electrode includes a grid corresponding to a plurality of individually addressable pixels.

31. The lens of claim 1, wherein the lens is configured to correct for non-conventional refractive error via selective movement of portions of the membrane.

32. The lens of claim 3, wherein the rigid optical element is configured to provide a toric correction (astigmatic optical power).

33. The lens of claim 1, wherein the membrane is configured to provide a toric correction (astigmatic optical power).

34. The lens of claim 1, wherein the lens is configured to change optical power to correct for far, intermediate, and near vision correction needs of a wearer.

35-37. (canceled)

38. The lens of claim 1, wherein the lens is at least one of a spectacle lens, a contact lens, an intra-ocular lens, a camera lens, a lens for a medical device, or a lens for an optical scanner.
